(19) United States
(12) Patent Application Publication
(10) Pub. No.: US 2004/0082986 A1
(43) Pub. Date: Apr. 29, 2004

(54) Unitary medical electrical lead and methods for making and using same

(76) Inventors: Randy Westlund, Minneapolis, MN (US); Christopher P. Knapp, Ham Lake, MN (US)

Correspondence Address: CRAWFORD PLLC, 1270 Northland Drive, Suite 390, St. Paul, MN, 55120, US

(21) Appl. No.: 10/278,731

(22) Filed: Oct. 23, 2002

(51) Int. Cl. A61N 1/05

(52) U.S. Cl. 607/122

(57) ABSTRACT

A unitary lead body of a medical electrical lead is formed of a flexible material and has a substantially cylindrical shape. An outer surface of the unitary lead body defines an outer surface of the lead, thereby obviating the need for a separate outer surface or sheath. The unitary lead body includes one or more longitudinally extending cavities defining a number of lumens. One or more electrical conductors are disposed within at least one of the lumens. One or more electrodes are respectively coupled to the electrical conductors. The unitary lead body is preferably formed by use of an extrusion process.

### FIELD OF THE INVENTION

The present invention relates generally to implantable medical electrical leads and, more particularly, to a medical electrical lead that employs a unitary lead body formed of a flexible material. The present invention further relates to methods of making and using a medical electrical lead having a unitary lead body.

### BACKGROUND OF THE INVENTION

Implantable cardiac stimulators are effective devices for treating patients with cardiac rhythmic dysfunction. A typical ICD includes a pulse generator and an electrical lead system with electrodes that engage cardiac tissue. A typical ICD implantation procedure generally takes about two hours and is relatively low risk, as it rarely requires open heart surgery. Usually, one to two lead wires are placed through a large vein in the chest and advanced down to the inside of the heart. The lead wires are then connected to the pulse generator, which is placed in a pocket under the skin of the patient.

Accessing the left side of the heart during a lead implantation procedure is often challenging, particularly when navigating cardiac structures of a diseased heart. This challenge is made more complicated when multiple leads or multiple conductor leads are to be advanced into the heart, and particularly the left atrium and/or left ventricle. By way of example, an ICD implemented to perform cardiac resynchronization therapies for congestive heart failure patients can often implicate implantation of electrodes within three or four chambers of the heart, including the left atrium and left ventricle. Those skilled in the art readily appreciate the difficulties of safely and timely accessing these left side cardiac structures during an implantation procedure.

There is a general need for smaller leads that provide adequate payload space to accommodate a sufficient number of conductors to support the functionality of a given ICD or other cardiac pacing or monitoring device. There is a more specific need for smaller leads manufactured with more volume efficient components, which is of particular interest in the design and implementation of left sided cardiac lead systems. The present invention fulfills these and other needs, and addresses other deficiencies of prior art implementations and techniques.

### SUMMARY OF THE INVENTION

The present invention is directed to a unitary medical electrical lead and methods of making and using same. According to one embodiment, a unitary lead body is formed of a flexible material and has a substantially cylindrical shape. An outer surface of the unitary lead body defines an outer surface of the lead, thereby obviating the need for a separate outer surface or sheath. The unitary lead body includes one or more longitudinally extending cavities defining a number of lumens. One or more electrical conductors are disposed within at least one of the lumens. One or more electrodes are respectively coupled to the electrical conductors.

The unitary lead body can be configured to include a single cavity defining at least two lumens or multiple cavities defining a multiplicity of lumens. An open primary lumen of the unitary lead typically includes a lubricious surface, such as a lubricious coating or tube. The open primary lumen can be adapted to receive a coil conductor. The open primary lumen can also be adapted to receive a stylet, a guide wire, or a sensor catheter, for example.

One or more secondary lumens of the unitary lead may each include a lubricious surface, such as an electrically insulating surface or tube. A given secondary lumen may be configured as an open lumen or a closed lumen. An open secondary lumen typically accommodates one or more electrical conductors, such as cable, wire, or coil conductor. A closed secondary lumen is typically adapted to receive a stylet. A secondary lumen can also be filled with a filler body or filler material to achieve a desired lead stiffness profile.

In accordance with another embodiment of the present invention, a unitary medical electrical lead can be fabricated using various extrusion techniques. The unitary lead can be formed from extruded tubing and include a primary lumen and one or more satellite lumens. Primary and satellite conductors can be fitted into respective primary and satellite lumens during fabrication or strung while the unitary tube structure is chemically expanded. A co-extrusion technique can also be employed to extrude the unitary lead body from one material, such as silicone or polyurethane, and concurrently coat the conductors with an electrically insulating material, such as ETFE (ethylene tetrafluoroethylene) or PTFE (polytetrafluoroethylene). A lubricious liner can also be co-extruded during the unitary lead body extrusion process. Further, a lubricious tube may be installed as part of the extrusion process, and the tube can be coated with a lubricious material via a co-extrusion technique.

According to a further embodiment, a method of implanting a unitary medical electrical lead into a cardiac structure of a patient's heart involves use of a guiding catheter for longitudinally guiding the unitary lead through cardiac vessels and structures. The unitary lead is of a configuration previously described. A guide wire is employed for longitudinally guiding the lead. The implantation method involves inserting the guiding catheter into a chamber of the patient's heart via an access vessel. The guide wire is inserted through the guiding catheter into the cardiac structure. The lead is then inserted through the guiding catheter and over the guide wire via the open primary lumen of the lead to implant the lead within or on the cardiac structure.

A secondary lumen of the lead can be configured to include a closed distal portion and an open proximal portion. The open proximal portion of the secondary lumen is configured to receive a stylet. The method of lead implantation further involves using the stylet to assist in directing the lead to the cardiac structure. A secondary lumen can also be configured as an open lumen for receiving a sensor catheter. The method of lead implantation can further involve using a sensor provided with the sensor catheter to assist in directing the lead to the cardiac structure.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

FIG. 1 is a partial view of one embodiment of an implantable medical device with a cardiac lead system extending into atrial and ventricular chambers of a heart, the cardiac lead system having a unitary lead body in accordance with the principles of the present invention;

FIG. 2 is a cross-sectional depiction of a conventional medical electrical lead having a two-part lead body construction and standard web dimensions between electrical conductors to provide the requisite electrical insulation therebetween;

FIG. 3 is a cross-sectional depiction of a medical electrical lead in accordance with an embodiment of the present invention having a unitary lead body construction which eliminates web spacing between electrical conductors otherwise needed to provide the requisite electrical insulation therebetween;

FIG. 4 is a cross-sectional depiction of the inner portion of a medical electrical lead in which lumens provided therein are separated by an insulating web according to conventional approaches;

FIG. 5 is a cross-sectional view of a medical electrical lead in accordance with an embodiment of the present invention, the unitary construction of the lead providing for an increase in volumetric payload efficiency;

FIG. 6 is a cross-sectional depiction of the inner portion of a medical electrical lead in which lumens provided therein are separated by an insulating web according to conventional approaches;

FIG. 7 is a cross-sectional view of a medical electrical lead in accordance with an embodiment of the present invention, the unitary construction of the lead providing for an increase in the number of available lumens without an increase in lead size relative to a conventional lead implementation of equal size;

FIG. 8 is a cross-sectional view of a medical electrical lead in accordance with an embodiment of the present invention, the unitary lead having a single cavity which defines multiple lumens;

FIG. 9 is a cross-sectional view of a medical electrical lead in accordance with an embodiment of the present invention, the unitary lead having multiple lumens separated from one another;

FIG. 10 is a cross-sectional view of a medical electrical lead in accordance with an embodiment of the present invention, the unitary lead including an electrical conductor provided within a primary lumen;

FIG. 11 is a cross-sectional view of a medical electrical lead in accordance with an embodiment of the present invention, the unitary lead including an electrical conductor provided within a primary lumen and various conductors or other elements provided within a multiplicity of secondary or satellite lumens;

FIG. 12 is a view of a medical electrical lead in accordance with an embodiment of the present invention, the unitary lead including various conductors provided within primary and satellite lumens;

FIG. 13 is a view of a medical electrical lead in accordance with an embodiment of the present invention, the unitary lead including an electrical conductor provided within a primary lumen and a stylet provided within a closed satellite lumen;

FIG. 14 is a view of a medical electrical lead in accordance with an embodiment of the present invention, the unitary lead including an electrical conductor provided within a primary lumen, various conductors or other elements provided within a multiplicity satellite lumens, and a stiffening material or body provided with a satellite lumen; and

FIGS. 15-16 show an embodiment of a tool useful for installing individual conductors within individual lumens during lead fabrication.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF VARIOUS EMBODIMENTS

In the following description of the illustrated embodiments, references are made to the accompanying drawings which form a part hereof, and in which is shown by way of illustration, various embodiments in which the invention may be practiced. It is to be understood that other embodiments may be utilized, and structural and functional changes may be made without departing from the scope of the present invention.

The embodiments of the present system illustrated herein are generally described in connection with an implantable cardiac defibrillator (ICD), which may operate in numerous pacing modes known in the art. A medical electrical lead of the present invention may also be implemented for use with other implantable cardiac devices that sense electrical activity, such as pacemakers, cardiac re-synchronizers, and cardiac monitors, for example. Further, a medical electrical lead of the present invention may be implemented for use with implantable medical devices that sense electrical activity within other organs or parts of the body.

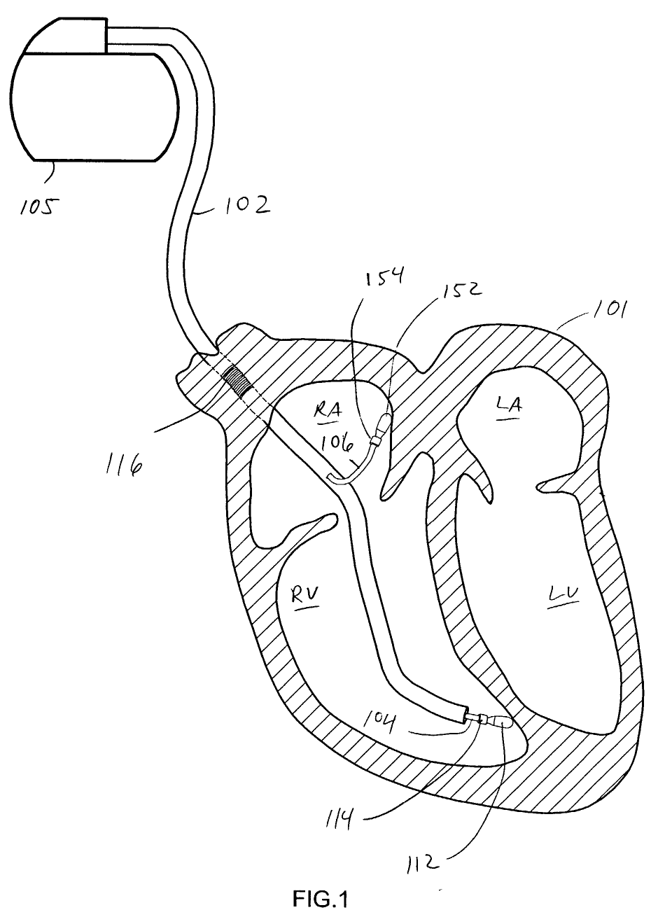

Referring now to FIG. 1 of the drawings, there is shown one embodiment of a medical device system which includes a medical electrical lead of the present invention. The medical device system is shown to include an implantable cardiac defibrillator 105 electrically and physically coupled to an intracardiac lead system 102. As will be described in detail below, the intracardiac lead system 102 includes a unitary lead body of the present invention. The intracardiac lead system 102 is implanted in a human body with portions of the intracardiac lead system 102 inserted into a heart 101. The intracardiac lead system 102 is used to detect and analyze electric cardiac signals produced by the heart 101 and to provide electrical energy to the heart 101 under certain predetermined conditions to treat anomalous cardiac activity, such as arrhythmias, for example. In an embodiment in which only monitoring of cardiac activity is performed, the intracardiac lead system 102 need not provide for the production of electrical energy to stimulate the heart 101.

The intracardiac lead system 102 includes one or more sense/pace electrodes. The intracardiac lead system 102 can also include one or more intracardiac defibrillation electrodes. In the particular embodiment shown in FIG. 1, the intracardiac lead system 102 includes a ventricular lead system 104 and an atrial lead system 106. The ventricular lead system 104 includes an SVC-coil 116, an RV-coil 114, and an RV-tip electrode 112. The RV-coil 114, which is also referred to as an RV-ring electrode and may be implemented as a non-coil electrode, is spaced apart from the RV-tip electrode 112, which is a pacing electrode. In one embodiment, the ventricular lead system 104 is configured as an integrated bipolar pace/shock lead. The atrial lead system 106 includes an A-tip electrode 152 and an A-ring electrode 154.

In this configuration, the intracardiac lead system 102 is positioned within the heart 101, with a portion of the atrial lead system 106 extending into the right atrium (RA) and portions of the ventricular lead system 104 extending into the right atrium and right ventricle (RV). In particular, the A-tip electrode 152 and A-ring electrode 154 are positioned at appropriate locations within the right atrium. The RV-tip electrode 112 and RV-coil 114 are positioned at appropriate locations within the right ventricle. The SVC-coil 116 is positioned at an appropriate location within the right atrium chamber of the heart 101 or a major vein leading to the right atrium chamber of the heart 101. The RV-coil 114 and SVC-coil 116 depicted in FIG. 1 are defibrillation electrodes.

The ventricular and atrial lead systems 104,106 include conductors for communicating sense, pacing, and defibrillation signals between the cardiac defibrillator 105 and the electrodes and coils of the lead systems 104, 106. A wide variety of conductor configurations can be employed, including single or multiple conductive element configurations, spring or coil conductive elements, cables, single or multiple strand wires, and the like.

As is shown in FIG. 1, the ventricular lead system 104 includes a conductor for transmitting sense and pacing signals between the RV-tip electrode 112 and an RV-tip terminal within the cardiac defibrillator 105. A conductor of the ventricular lead system 104 transmits sense signals between the RV-coil or ring electrode 114 and an RV-coil terminal within the cardiac defibrillator 105. The ventricular lead system 104 also includes a conductor for transmitting sense and defibrillation signals between an SVC-coil terminal of the cardiac defibrillator 105 and the SVC-coil 116. The atrial lead system 106 includes conductors for transmitting sense and pacing signals between A-tip and A-ring terminals of the cardiac defibrillator 105 and A-tip and A-ring electrodes 152 and 154, respectively.

Additional pacing and defibrillation electrodes may also be included in the intracardiac lead system 102 to allow for various sensing, pacing, and defibrillation capabilities. For example, the intracardiac lead system 102 may include endocardial pacing and cardioversion/defibrillation leads (not shown) that are advanced into the coronary sinus and coronary veins to locate the distal electrode(s) adjacent to the left ventricle or the left atrium. The distal end of such coronary sinus leads is advanced through the superior vena cava, the right atrium, the valve of the coronary sinus, the coronary sinus, and into a coronary vein communicating with the coronary sinus, such as the great vein. Other intracardiac lead and electrode arrangements and configurations known in the art are also possible and considered to be within the scope of the present system.

A medical electrical lead according to the present invention, which may be implemented in a manner discussed above, provides several advantages not realizable with conventional leads. One advantage concerns a significant reduction in total lead diameter due to the elimination of, or significant reduction in, an electrically insulating web of material otherwise needed between the various electrical conductors of a conventional lead. Another advantage concerns the elimination of a separate outer sleeve material which is required in conventional two-part lead constructions.

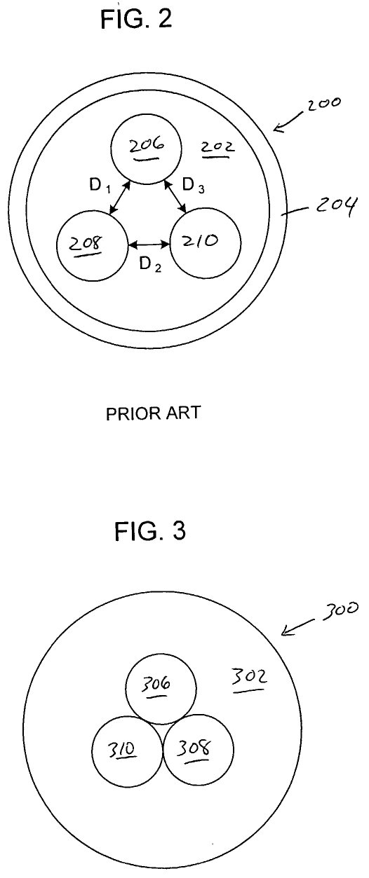

A unitary lead body construction according to the present invention advantageously provides maximum opportunity to reduce the overall diameter of a multiple conductor medical electrical lead and to maximize volumetric payload capacity within the unitary lead body. For example, a significant reduction in lead diameter in comparison to a conventional lead can be realized while maintaining the same number of conductors and/or lumens as the conventional lead. Further, a lead of the present invention can include a greater number of conductors and/or lumens as compared to a conventional lead having the same diameter. These and other advantages of a unitary medical electrical lead of the present invention, and methods of making and using same, can be appreciated from the following discussion concerning FIGS. 2-16.

Turning now to FIG. 2, there is illustrated a cross-sectional depiction of a conventional medical electrical lead 200 having a two-part lead body construction. In particular, the conventional medical electrical lead 200 includes an inner core member 202 and separate outer insulating tubing or sheath 204. The material used to fabricate the inner core member 202 is typically different from that of the outer insulting tubing 204. For example, the inner core member 202 is often fabricated from silicone rubber and the outer insulating tubing 204 is often fabricated from polyurethane.

As can further be seen in FIG. 2, electrical conductors 206, 208, 210 are separated a sufficient distance D1, D2, D3 from one another in order to provide the requisite electrical insulation between the respective conductors. The spacing dimensions D1, D2, D3 are determined based on a number of design considerations, including conductor diameters and materials, current and voltage requirements of the conductors, and the electrical insulating properties of the inner core member material. By way of example, typical spacing dimensions D1, D2, D3 can range between 5 and 12 thousandths of an inch.

Conventional leads must therefore be designed to accommodate an insulating web of sufficient size to meet the electrical insulating requirements of the lead's conductors. The presence and size of the insulating web of material provided between the conductors 206, 208, 210 can thus significantly increase the overall size (i.e., outer diameter) of the lead 200.

Moreover, according to a conventional lead design, the bulk material of the inner core member 202 must be selected to meet a host of mechanical, biological, and electrical requirements. Since no single material can provide optimal characteristics for meeting all mechanical, biological, and electrical requirements, a balance or compromise is typically struck to best meet the various design requirements. For example, a given material may provide excellent mechanical performance for a given lead design, in which case the electrical properties of such material represent design constraints that dictate the amount of insulating web material the must be present between the lead's electrical conductors. The inability to select particular materials to optimize mechanical and electrical performance limits the designer's ability to reduce lead diameters or increase payload volumes without increasing lead size.

In contrast to the conventional lead construction depicted in FIG. 2, a unitary lead body construction of the present invention advantageously eliminates the need for an insulating web of material between lead conductors. Moreover, a lead body constructed in accordance with the present invention provides for the use of materials having optimal electrical and mechanical characteristics, without having to comprise between such characteristics. For example, a web of bulk insulating material of a conventional lead having a thickness of between 5 to 12 thousandths of an inch between adjacent conductors can be eliminated by use of a unitary lead body construction of the present invention and a 2 thousandths of an inch coating of insulating material around each conductor. A lead designer can select optimal materials for the lead body core and optimal materials to provide the requisite electrical insulation between conductors of the lead.

FIG. 3 is a cross-sectional depiction of a medical electrical lead 300 implemented in accordance with an embodiment of the present invention. The medical electrical lead 300 has a unitary lead body construction which eliminates web spacing between electrical conductors which is otherwise needed to provide the requisite electrical insulation therebetween. For example, the spacing dimensions D1, D2, D3 required to provide sufficient electrical insulation for electrical conductors 206, 208, 210, as shown in FIG. 2, can be wholly eliminated, as is shown in FIG. 3.

The electrical conductors 306, 308, 310 of FIG. 3 are shown as having essentially no interposing insulating web. Rather, electrical conductors 306, 308, 310 are each provided with an insulating coating (e.g., pre-coated or co-extruded) formed of a material selected to have optimal electrical properties. The bulk material of the unitary lead body 302 is formed of a material that can be selected to provide optimal mechanical and biological properties. As such, a unitary lead fabricated in accordance with the principles of the present invention provides the design freedom to select optimal materials to achieve the mechanical, electrical, and biological requirements of a given lead design, while minimizing lead size and maximizing volumetric payload efficiency.

For example, a lead designer can select a coating surface material for the electrical conductors of the lead that has a dielectric constant substantially greater than a dielectric constant of the flexible material of the unitary lead body. The lead designer may, for example, select ETFE as an electrical insulator (tube or coating) for the electrical conductors, and select silicone or polyurethane to form the unitary lead body.

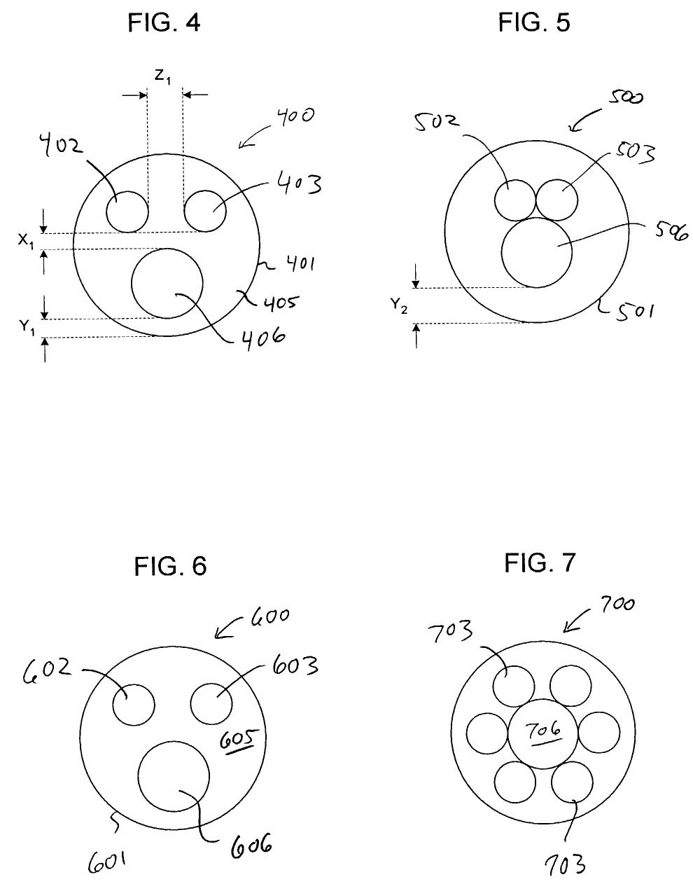

FIGS. 4 and 5 illustrate the significant increase in useable payload space within a medical electrical lead of the present invention in comparison to a conventional lead of equal outer diameter. The lead 400 shown in FIG. 4 is of a conventional design, and includes a web of insulating bulk material that separates electrical conductors 402, 403, and 406. A web spacing, Z₁, is required between conductors 402 and 403, and a web spacing, X₁, is required between conductor 406 and conductors 402, 403. It is noted that lead 400 includes a outer tube 401 formed from a material different from that of the lead's inner core 405.

The medical electrical lead 500 shown in FIG. 5 is implemented in accordance with the present invention, and has an outer diameter equal to that of lead 400 shown in FIG. 4. The medical electrical lead 500 has a unitary body 501 which includes three lumens. Electrical conductors 502, 503, 506 are respectively disposed in the lumens. As can be readily seen in FIG. 5, the web spacings, Z₁. and X₁, between conductors 402, 403, and 406 are eliminated with respect to conductors 502, 503, and 506 in the unitary lead implementation of FIG. 5. This elimination of web spacing between conductors provides for an effective increase in useable space within the lead body 501, as can be seen by the increase in the outer surface-to-conductor spacing dimension Y₂ of FIG. 5 relative to Y₁shown in FIG. 4.

FIGS. 6 and 7 illustrate the advantage of increased volumetric payload efficiency achievable when implementing a medical electrical lead in accordance with the present invention. FIG. 6 illustrates a conventional medical lead 600 which includes a web of insulating bulk material 605 that separates three electrical conductors 602, 603, and 606. Lead 600 has an outer surface 601 formed of a material different from that of the inner core 605. The outer diameter of lead 600 is assumed equal to that of unitary lead 700 shown in FIG. 7. Lead 600, in this illustrative example, is depicted to include the maximum number of three electrical conductors suitable for a lead of this size.

The unitary lead 700, as is readily seen in FIG. 7, can accommodate several more electrical conductors/lumens 703, 706 within a lead body having the same outer diameter as that of FIG. 6. As shown, unitary lead 700 accommodates a primary conductor/lumen 706 equal in diameter to conductor/lumen 606 of conventional lead 600. Further, unitary lead 700 can accommodate six to eight satellite or secondary conductors/lumens 703 (six of which are shown in FIG. 7), whereas conventional lead 600 can only safely accommodate two of such secondary conductors/lumens 602, 603.

One skilled in the art will readily appreciate the many advantages offered by a unitary lead 700 of the present invention, including an increased number of lumens without an increase in overall lead size, or a reduction in overall lead size without reducing the number of available lumens. The increased number of available lumens provided by a unitary lead of the present invention provides an opportunity to employ a multiplicity of lead components without increasing overall lead size, such components including, for example, various types of electrical conductors (e.g., coils, cables, wires), sensor catheters (e.g., pressure, temperature, oxygen saturation, optical, imaging, and Doppler sensor catheters), stylets, guide wires, finishing wires, and stiffening members.

By way of example, a unitary medical electrical lead of the present invention which includes two lumens can readily be formed to have an overall diameter of 4 French or less. By way of further example, a unitary medical electrical lead of the present invention which includes a multiplicity of lumens to accommodate eight conductors/electrodes for congestive heart failure pacing and sensing can readily be formed to have an overall diameter of 12 French or less.

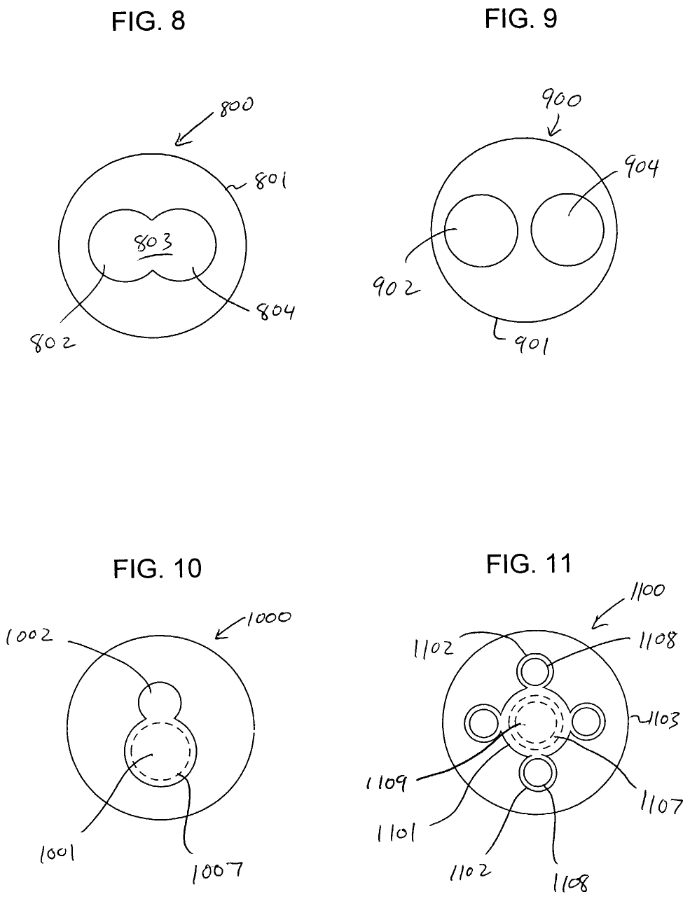

Turning now to FIG. 8, there is illustrated an embodiment of a medical electrical lead implemented in accordance with the principles of the present invention. According to this embodiment, the medical electrical lead includes a unitary lead body 800 formed of a flexible material and has a substantially cylindrical shape. The lead body 800 has an outer surface 801 which defines an outer surface of the lead. As such, the lead body 800 does not require a separate outer tubing or sheath as does a conventional lead body.

The unitary lead body 800 is formed to include one or more longitudinally extending cavities 803 that define a number of lumens. As shown in FIG. 8, the unitary lead body 800 includes a single cavity 803 configured to define two lumens 802, 804. One or both of the lumens 802, 804 can be formed as an open lumen. Depending on a particular lead design, one or more electrical and/or optical conductors can be disposed within at least one of the lumens 802, 804. At least some of the conductors are electrically coupled to various electrodes for sensing cardiac activity and delivering pacing, cardioverting, and/or defibrillating energy to the heart. Other conductors can be disposed within one or both of the lumens 802, 804 and coupled to various types of sensor elements.

FIG. 9 illustrates another embodiment of a medical electrical lead of the present invention. According to this embodiment, a unitary lead body 900 is formed to include two or more lumens that are separated from one another by the bulk material of the lead body 900. As show in FIG. 9, the unitary lead body 900 includes two such lumens, wherein a first lumen 902 is separated from a second lumen 904 by the bulk material of the lead body 900. As in the case of the lead body 800 shown in FIG. 8, the lead body 900 of FIG. 9 has an outer surface 901 which defines an outer surface of the lead, thereby obviating the need for a separate outer tubing or sheath.

FIG. 10 illustrates a further embodiment of a medical electrical lead of the present invention. According to this embodiment, a unitary lead body 1000 is formed to include a single longitudinally extending cavity that defines two lumens 1001 and 1002. Lumen 1001 is preferably an open lumen that receives a coil conductor 1007. Lumen 1001 can be provided with a lubricious tube or coating, such as an electrically insulating lubricious coating. The satellite lumen 1002 can be configured to accommodate a cable conductor, such as a cable conductor that connects to a pace/sense electrode.

By way of example, the unitary lead body 1000 can be formed to have an outer diameter of about 0.0675 inches. The diameter of lumen 1001 can be about 0.045 inches. The wall surface of lumen 1001 can be provided with an ETFE or PTFE coating or tube, such as an ETFE coating having a thickness ranging between 0.001 inches and 0.003 inches. Alternatively, or in addition, the coil conductor 1007 can be provided with an inner electrically insulating coating or tube, and preferably includes an electrically insulating lubricious inner surface coating, such as ETFE or PTFE. Coil conductor 1007 can have an inner diameter of about 0.025 inches, an outer diameter of about 0.043 inches, and include 0.005 inch filars and an ETFE coating having a thickness of about 0.002 inches in this particular example.

The coil conductor 1007 defines a central lumen that can accommodate passage of a guide wire or stylet, for example. The satellite lumen 1002 can be configured to accommodate a cable conductor. The satellite lumen 1002 can have a diameter of about 0.0075 inches. The cable conductor is preferably coated with an electrically insulating coating, such as ETFE or PTFE. The satellite lumen 1002 can alternatively, or in addition, be coated with a lubricious material, such as a lubricious insulating coating, or fitted with a suitable lubricious tube (e.g., ETFE or PTFE tube). The satellite lumen 1002 can also be configured to accommodate a coil conductor, a single conductive wire, one or more optical fibers, or a combination of such conductors with appropriate electrical insulation provided.

FIG. 10 illustrates a volume efficient unitary lead construction in which a primary lumen 1001 is formed proximate a satellite lumen 1002 such that no web of core material is needed the insulate electrically conductive elements deployed in lumens 1001 and 1002. This increase in volumetric payload efficiency can be exploited by a reduction in overall lead size, provision of a greater mass of lead core material between the lumens 1001, 1002 and outer surface 1003 of the lead body 1000 for enhanced abrasion resistance, or a balance of these design considerations. By way of example, the outer surface 1003 of the lead body may be separated from the lumens 1001, 1002 by about 0.0075 inches (i.e., an outer wall section of the unitary lead body 1000 of about 0.0075 inches).

Another embodiment of a multiple lumen unitary lead body 1100 is illustrated in FIG. 11. According to this embodiment, the unitary lead body 1100 is formed to include a primary lumen 1101 and several satellite lumens 1102. In the particular configuration shown in FIG. 11, a central cavity of the lead body 1100 is formed to include one primary lumen 1101 and four satellite lumens 1102. The primary lumen 1101 is provided with a coil conductor 1107 which defines a central lumen 1109. The coil conductor 1107 is preferably coated with an electrically insulating lubricious material as previously discussed. The central lumen 1109 provides for the deployment of a guide wire, sensor, stylet or other device or member that can be displaced longitudinally within the open passageway of the central lumen 1109.

The satellite lumens 1102 can be formed to accommodate various types of elements 1108, such as cables, wires, coil conductors, sensors, stylets and the like. One or more of the satellite lumens 1102 can also be filled with a material that provides constant or variable stiffness to the lead body 1100. For example, all or a portion(s) of a satellite lumen 1102 can be filled with a polymer material or filler of varying modulus that imparts a desired degree of stiffness to the lead body 1100. Different sections of a satellite lumen 1102 can be filled with the same or different stiffening material to achieve a desired stiffness profile along the length of the lead body 1100.

Alternatively, or in addition, a coil conductor deployed in the primary lumen 1101 (e.g., coil conductor 1107) or a coil conductor deployed in a satellite lumen 1102 can be wound with, or otherwise incorporate, different filars to achieve a desired lead body stiffness profile. According to another approach, lead body 1100 and, therefore, the satellite lumens 1102, can be formed using a variable extrusion technique, in which case the size, shape, and number of satellite lumens 1102 can be selectively formed and filled with a stiffening material or member to achieve a desired lead body stiffness profile. The primary and satellite lumens 1101, 1102 can be formed to have dimensions equal to those described in the embodiment in FIG. 10, with an overall lead diameter of about 0.075 inches to accommodate the additional satellite lumens 1102.

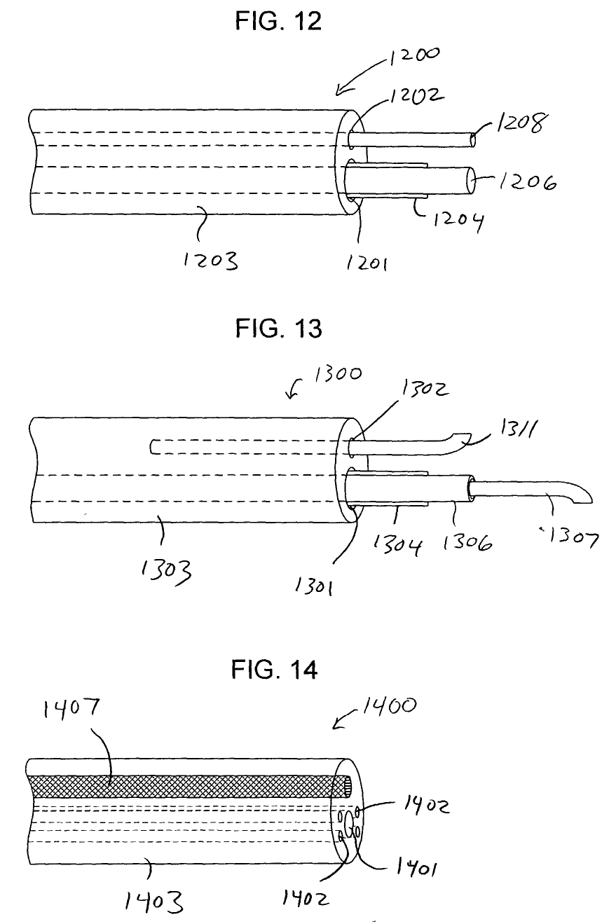

FIG. 12 illustrates an embodiment of a unitary medical electrical lead according to the present invention. In this embodiment, the unitary lead 1200 includes a unitary lead body 1203 having a primary lumen 1201 and a satellite or secondary lumen 1202. It is noted that the primary lumen 1201 and satellite lumen 1202 are shown defined within separate cavities of unitary lead body 1203, but can alternatively be defined within a common cavity. A coil conductor 1204 extends longitudinally within the primary lumen 1201. In one configuration, a lubricious tube 1206, such as an ETFE or PTFE tube, is situated within the central opening of the coil conductor 1204. The interior of the lubricious tube 1206 defines a central lumen within which various members, devices, and catheters can be longitudinally displaced as previously discussed. In another configuration, the coil conductor 1204 can be coated with a lubricious material 1206, such as ETFE or PTFE. The satellite lumen 1202 is shown to accommodate a cable or wire conductor 1208.

Another embodiment of a unitary medical electrical lead according to the present invention is illustrated in FIG. 13. According to this embodiment, the unitary lead 1300 includes a unitary lead body 1303 having an open primary lumen 1301 and a closed satellite lumen 1302. The primary lumen 1301 is shown to accommodate a coil conductor 1304 and a lubricious tube 1306, which can alternatively be representative of a lubricious coating as in FIG. 12. The interior of the lubricious tube 1306 defines a central lumen within which a guide wire 1307 is shown deployed for longitudinal displacement within the central lumen.

The satellite lumen 1302 has an opening located at a proximal end of the lead body 1303 and terminates within the lead body 1303 proximal to the distal end of the unitary lead 1300 so as to define a closed lumen. The closed satellite lumen 1302 can be configured to accommodate a variety of members, including a stylet 1311. The closed satellite lumen 1302 allows a clinician to easily attach and remove a variety of stylets 1311 for accessing a variety of cardiac structures and vessels. For example, a stylet 1311 having a shape optimized for accessing the coronary sinus can be inserted into the closed satellite lumen 1302.

FIG. 14 illustrates yet another embodiment of a unitary medical electrical lead according to the present invention. According to this embodiment, the unitary lead 1400 includes a unitary lead body 1403 having an open primary lumen 1401 and several satellite lumens 1402. The primary lumen 1401 can accommodate a coil conductor and a lubricious tube or coating as previously described, so as to define a central lumen within which a guide wire, sensor catheter, stylet or other member/device can be deployed for longitudinal displacement therein.

As shown, unitary lead body 1403 is formed to include four satellite lumens 1402 which are intended to accommodate electrically conductive wires, cables, coil conductors or other members, catheters, or devices as previously described. Unitary lead body 1403 further includes a fifth satellite lumen 1407 which is designed to receive a stiffening material or member 1405. The stiffening material or member can provide constant or variable stiffness along the length of the unitary lead body 1403 as previously described. It is noted that the primary lumen 1401 and satellite lumens 1402, 1407 are shown defined within separate cavities of unitary lead body 1403. It is understood that all or some of these lumens 1401,1402, 1407 can be defined within one or more common cavities.

In accordance with another embodiment of the present invention, a unitary medical electrical lead can be fabricated using various extrusion techniques. The unitary lead can be formed from extruded tubing so as to define a primary lumen and one or more satellite lumens. Primary and satellite conductors can be fitted into place during fabrication or strung while the unitary tube structure is chemically expanded, such as by use of freon gas as is known in the art. A co-extrusion technique can also be employed to extrude the unitary lead body from one material (e.g., silicone or polyurethane) and concurrently coat the conductors with an electrically insulating material (e.g., ETFE or PTFE). A lubricious liner can also be co-extruded during the unitary lead body extrusion process. Further, a lubricious tube may be installed as part of the extrusion process, and the tube can be coated with a lubricious material via a co-extrusion technique.

In one approach, the electrical conductors (e.g., cables, wires, coil conductors) to be installed in the primary and satellite lumens can be pre-coated with a suitable electrically insulating and, if desired, lubricious material, such as ETFE or PTFE. The electrical conductors can then be installed as described above.

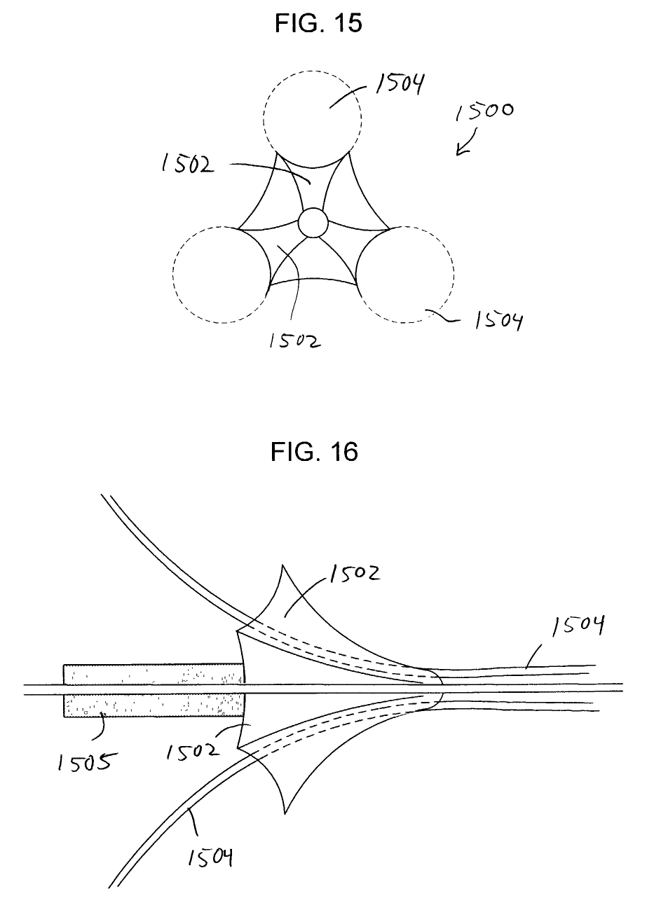

To increase conductor installation efficiency, a installation tool, such as tool 1500 shown in FIGS. 15 and 16, can be employed during lead fabrication. After a set of conductors 1504 have been situated within a common, multiple lumen cavity of the extruded lead body, tool 1500 can be advanced longitudinally through the common cavity via displacement of shaft 1505 to separately install each conductor 1504 into its respective lumen.

Installation tool 1500 includes several tapered, wedge-shaped ramps 1502 which correspond in number to the number of electrical conductors 1504 to be installed into the lead body. Each of the ramps 1502 has a shape specifically configured to accommodate the curvature of a particular electrical conductor 1504. The tool 1500 is displaced longitudinally from a proximal opening to a distal opening of the lead body cavity. As the tool 1500 is advanced through the lead body cavity, each ramp 1502 engages its corresponding conductor 1504 and forces the conductor into its respective lumen. The tool 1500 and shaft 1505 are removed via the distal opening of the lead body cavity.

According to one particular fabrication approach, a medical electrical lead of the present invention is formed by extruding a polymeric material to form a unitary lead body having one or more longitudinally extending cavities defining a number of lumens. The lumens typically include at least one open primary lumen. An outer surface of the unitary lead body defines an outer surface of the lead, thus obviating the need for a separate outer layer of material. One or more electrical conductors are installed within at least one of the lumens, to which one or more electrodes are respectively connected.

The fabrication approach can further involve extruding an electrically insulating material into at least one of the lumens or coating one or more of the electrical conductors with an electrically insulating material while extruding the polymeric material of the lead body. As discussed above, a lubricious material, such as an electrically insulating material, can be co-extruded in at least one of the lumens.

A unitary medical electrical lead of the present invention may be used to facilitate a variety of sensing, pacing, cardioverting, and defibrillating functions for single and multiple chamber applications. For example, a single unitary lead of the present invention can be configured for deployment in various multiple chamber configurations, including a first configuration for the right atrium and right ventricle, a second configuration for the right atrium and left atrium, a third configuration for the left atrium and left ventricle, and a fourth configuration for the right atrium, left atrium, and left ventricle.

As was discussed previously, a medical electrical lead implemented in accordance with the present invention provides for an increased number of lumens available for a variety of uses without an increase in overall lead size. This affords the clinician a greater number of tools available during lead implantation, particularly in connection with lead implantation within the left atrium and left ventricle via the coronary sinus. Such tools include guide wires for over-the-wire implantation procedures, for example.

In accordance with another embodiment of the present invention, a method of implanting a unitary medical electrical lead into a cardiac structure of a patient's heart involves use of a guiding catheter for longitudinally guiding the unitary lead through cardiac vessels and structures. The unitary lead is of a configuration previously described. A guide wire is employed for longitudinally guiding the lead. The implantation method involves inserting the guiding catheter into a chamber of the patient's heart via an access vessel. The guide wire is inserted through the guiding catheter into the cardiac structure. The lead is then inserted through the guiding catheter and over the guide wire via the open primary lumen of the lead to implant the lead within or on the cardiac structure.

In one configuration, the lead can include one or more electrical conductors for implanting within or on at least two chambers of the patient's heart. In another configuration, the lead includes one or more electrical conductors for implanting within or on three chambers of the patient's heart, such as the right atrium, left atrium, and left ventricle. The cardiac structure can also constitute a cardiac vessel, such as the coronary sinus or other cardiac vessel.

According to a further enhancement, a secondary lumen of the lead can be configured to include an open proximal portion and a closed distal portion. The open proximal portion of the secondary lumen receives a stylet. The method of lead implantation further involves using the stylet to assist in directing the lead to the cardiac structure. A secondary lumen can also be configured as an open central lumen for receiving a sensor catheter. The method of lead implantation can further involve using a sensor provided with the sensor catheter to assist in directing the lead to the cardiac structure.

Various modifications and additions can be made to the preferred embodiments discussed hereinabove without departing from the scope of the present invention. Accordingly, the scope of the present invention should not be limited by the particular embodiments described above, but should be defined only by the claims set forth below and equivalents thereof.

What is claimed is:

1. A medical electrical lead, comprising:
a unitary lead body formed of a flexible material and having a substantially cylindrical shape, an outer surface of the unitary lead body defining an outer surface of the lead, the unitary lead body comprising one or more longitudinally extending cavities defining a plurality of lumens, the plurality of lumens comprising an open primary lumen;
one or more electrical conductors disposed within at least one of the lumens; and
one or more electrodes respectively coupled to the one or more electrical conductors.

2. The lead of claim 1, wherein the unitary lead body comprises a single cavity defining at least two lumens.

3. The lead of claim 1, wherein the open primary lumen comprises a lubricious surface.

4. The lead of claim 3, wherein lubricious surface is an electrically insulating surface.

5. The lead of claim 3, wherein a secondary lumen of the plurality of lumens comprises a lubricious surface.

6. The lead of claim 1, wherein the open primary lumen is adapted to receive a stylet.

7. The lead of claim 1, wherein the open primary lumen is adapted to receive a guide wire.

8. The lead of claim 1, wherein the open primary lumen is adapted to receive a sensor catheter.

9. The lead of claim 1, wherein the open primary lumen is adapted to receive the one or more electrical conductors.

10. The lead of claim 1, wherein the one or more electrical conductors comprises a coil conductor defining a central lumen, the open primary lumen adapted to receive the coil conductor.

11. The lead of claim 1, wherein the plurality of lumens comprises an open secondary lumen.

12. The lead of claim 11, wherein the one or more electrical conductors are respectively disposed in the open secondary lumen and the open primary lumen.

13. The lead of claim 1, wherein a secondary lumen of the plurality of lumens comprises a closed distal portion and an open proximal portion.

14. The lead of claim 13, wherein the open proximal portion of the secondary lumen is adapted to receive a stylet.

15. The lead of claim 1, wherein:
the open primary lumen is adapted to receive a guide wire; and
a secondary lumen of the plurality of lumens comprises a closed distal portion and an open proximal portion, the open proximal portion of the secondary lumen adapted to receive a stylet.

16. The lead of claim 1, wherein the unitary lead body comprises the open primary lumen and a secondary lumen of the plurality of lumens, the unitary lead body having an outer diameter of less than about 4 French.

17. The lead of claim 1, wherein the plurality of lumens comprises a secondary lumen, the secondary lumen comprising a filler body or filler material.

18. The lead of claim 17, wherein a stiffness of the filler body or material can be varied to vary a stiffness of the lead.

19. The lead of claim 1, wherein the open primary lumen comprises a surface material having a dielectric constant substantially greater than a dielectric constant of the flexible material of the unitary lead body.

20. The lead of claim 1, wherein the unitary lead body is formed from silicone or polyurethane.

21. The lead of claim 1, wherein the unitary lead body is formed of an extruded polymeric material.

22. The lead of claim 1, wherein at least some of the one or more electrical conductors comprise an outer coating of an electrically insulating material.

23. A method of fabricating a medical electrical lead, comprising:
extruding a polymeric material to form a unitary lead body comprising one or more longitudinally extending cavities defining a plurality of lumens, the plurality of lumens comprising an open primary lumen, an outer surface of the unitary lead body defining an outer surface of the lead;
providing one or more electrical conductors within at least one of the lumens; and
connecting one or more electrodes respectively to the one or more electrical conductors.

24. The method of claim 23, further comprising co-extruding a lubricious material in at least one of the lumens.

25. The method of claim 23, further comprising co-extruding an electrically insulating material in at least one of the lumens.

26. The method of claim 23, further comprising extruding a lubricious coating into at least one of the lumens.

27. The method of claim 23, further comprising extruding an electrically insulating material into at least one of the lumens.

28. The method of claim 23, further comprising coating the one or more electrical conductors with an electrically insulating material while extruding the polymeric material.

29. The method of claim 23, wherein extruding the polymeric material comprises extruding a silicone or polyurethane material to form the unitary lead body, the method further comprising providing a lubricous tube within the open primary lumen.

30. The method of claim 23, wherein extruding the polymeric material comprises extruding a silicone or polyurethane material to form the unitary lead body, the method further comprising providing a lubricous tube within the open primary lumen.

31. The method of claim 30, wherein the lubricous tube is formed from PTFE.

32. The method of claim 23, wherein providing the one or more electrical conductors comprises fitting the one or more electrical conductors within the at least one of the lumens.

33. The method of claim 23, wherein providing the one or more electrical conductors comprises providing the one or more electrical conductors while extruding the polymeric material.

34. The method of claim 23, wherein providing the one or more electrical conductors further comprises:
fitting a set of electrical conductors within a cavity comprising at least two lumens; and
forcing one or more electrical conductors of the set of electrical conductors into each of the at least two lumens.

35. The method of claim 34, wherein forcing the one or more electrical conductors into each of the at least two lumens further comprises using a tool to force the one or more electrical conductors into each of the at least two lumens as the tool is moved longitudinally within the cavity.

36. A method of implanting a medical electrical lead into a cardiac structure of a patient's heart, comprising:
providing a guiding catheter for longitudinally guiding the lead, the lead comprising:
a unitary lead body formed of a flexible material and having a substantially cylindrical shape, an outer surface of the unitary lead body defining an outer surface of the lead, the unitary lead body comprising one or more longitudinally extending cavities defining a plurality of lumens, the plurality of lumens comprising an open primary lumen;
one or more electrical conductors disposed within at least one of the lumens; and
one or more electrodes respectively coupled to the one or more electrical conductors;
providing a guide wire for longitudinally guiding the lead;
inserting the guiding catheter into a chamber of the patient's heart via an access vessel;
inserting the guide wire through the guiding catheter into the cardiac structure; and
inserting the lead through the guiding catheter and over the guide wire via the open primary lumen to implant the lead within or on the cardiac structure.

37. The method of claim 36, wherein the lead comprises one or more electrical conductors for implanting within or on at least two chambers of the patient's heart.

38. The method of claim 36, wherein the lead comprises one or more electrical conductors for implanting within or on three chambers of the patient's heart.

39. The method of claim 36, wherein the three chambers of the patient's heart comprise a right atrium, a left atrium, and a left ventricle.

40. The method of claim 36, wherein the cardiac structure comprises a cardiac vessel.

41. The method of claim 36, wherein a secondary lumen of the plurality of lumens comprises a closed distal portion and an open proximal portion, the open proximal portion of the secondary lumen receiving a stylet, the method further comprising using the stylet to assist in directing the lead to the cardiac structure.

42. The method of claim 36, wherein a secondary lumen of the plurality of lumens comprises an open central lumen for receiving a sensor catheter, the method further comprising using a sensor provided with the sensor catheter to assist in directing the lead to the cardiac structure.

43. The method of claim 36, wherein the unitary lead body is formed of an extruded polymeric material.

44. The method of claim 36, wherein the unitary lead body is formed from silicone or polyurethane.
